(19) United States
(12) Patent Application Publication
(10) Pub. No.: US 2002/0103516 A1
(43) Pub. Date: Aug. 1, 2002

(54) Carotid sinus nerve stimulation for epilepsy control

(76) Inventors: Ravish Vinay Patwardhan, Birmingham, AL (US); Richard Shane Tubbs, Mt. Olive, AL (US)

Correspondence Address: Benjamin Aaron Adler, ADLER & ASSOCIATES, 8011 Candle Lane, Houston, TX, 77071, US

(21) Appl. No.: 09/957,566

(22) Filed: Sep. 20, 2001

Related U.S. Application Data
(60) Provisional application No. 60/233,876, filed on Sep. 20, 2000.

(51) Int. Cl. A61N 1/18

(52) U.S. Cl. 607/72

(57) ABSTRACT

The present invention describes a method of treating, controlling or preventing epilepsy, comprising the steps of attaching at least one electrode to at least one of a patient's carotid sinus nerves, and applying or modulating electric signals to or recording from at least one of the patient's carotid sinus nerves through the at least one electrode, so as to treat, control, or prevent epilepsy.

### CROSS-REFERENCE TO RELATED APPLICATION

This non-provisional patent application claims benefit of provisional patent application U.S. Ser. No. 60/233,876, filed Sep. 20, 2000, now abandoned.

### FEDERAL FUNDING LEGEND

This invention was produced in part using funds obtained through Grant HL-28429 from the National Heart, Lung and Blood Institute. Consequently, the federal government has certain rights in this invention.

### BACKGROUND OF THE INVENTION

1. Field of the Invention

The present invention relates generally to seizure control and neurological effects related to a specific cranial nerve branch stimulation. More specifically, the present invention relates to carotid sinus nerve stimulation for epilepsy control.

2. Description of the Related Art

An estimated 1% of the world's population suffers from epilepsy. Of these, approximately 30,000 patients each year suffer from medically intractable epilepsy, and alternative treatments are sought. Only 15% of patients will benefit from presently existing cerebral surgery techniques. Vagal nerve stimulation offers 20-61% seizure reduction (at 3-month or greater follow-up) according to most large studies, but is associated with some undesirable side effects (such as hoarseness, coughing, and dysautonomia; right-sided VNS in contraindicated due to potentially dangerous associated cardiac side effects. Of the approximately 50% of patients whose seizures are not well controlled by VNS (and therefore by most other available therapeutic options), as well as for those patients in whom VNS is difficult or contraindicated (e.g. due to prior scarring from radiation, cancer-related surgery, etc.), no other good therapeutic option exists.

The use of nerve stimulation to treat endocrine disorders is disclosed in U.S. Pat. No. 5,231,988, which states that electrical stimulation of the vagus nerve can treat the pancreatic disorders of hypoglycemia and diabetes mellitus. The use of nerve stimulation to treat and control migraine is disclosed in U.S. Pat. No. 5,215,086, which states that selective modulation of vagus nerve electrical activity can treat migraine symptoms. The use of a neurocybernetic prosthesis, including a pulse generator which generates electrical pulses, to control or prevent epileptic seizures, as well as treat various involuntary movements, is disclosed in U.S. Pat. Nos. 5,025,807, 4,867,164 and 4,702,254. These patents disclose electrical stimulation of the vagus nerve to treat involuntary movement disorders. U.S. Pat. No. 5,269,303 discloses the application of an electrical stimulating signal to the vagus nerve to treat symptoms of dementia, including cortical dementia, subcortical dementia and multi-infarct dementia. Neistadt and S. I. Schwartz in “Implantable Carotid Sinus Nerve Stimulator For Reversal of Hypertension” Surgical Forum Volume XVII, Proceedings of the 22nd Annual Session of the Forum on Fundamental Surgical Problems, 52nd Clinical Congress of the American College of Surgeons, San Francisco, Calif., Oct. 1966, Chapter V: Cardiovascular Problems, pp. 123-124, disclose an implantable carotid sinus nerve stimulator for reversal of hypertension. Parsonnet et al., in “Radio-Frequency Stimulation of the Carotid Baroreceptors in the Treatment of Hypertension” Surgical Forum supra, pp. 125-127, disclose that radiofrequency radiation used to stimulate a carotid baroreceptor was found to lower the systemic arterial blood pressure in dogs, but that responsiveness was lost in long-term nerve stimulation.

Vagal nerve stimulation (VNS) for control of medically refractory epilepsy was introduced by Zabara (U.S. Pat. No. 5,540,734), and has recently received approval for use in selected patients by the Food and Drug Administration. Although helpful as a last resort in many of these patients, VNS has been shown to b e limited in efficacy, with accompanying undesirable side effects (e.g. hoarseness, dysphagia, cough, and dysautonomia). VNS is also limited to the left side, since right-sided stimulation is associated with significant cardiac side effects. Patients who are candidates for VNS usually have failed all medical and other available surgical techniques. A patient presently failing VNS often has no other good treatment option available.

A more desirable technique would offer minimal invasiveness as with VNS, with improved efficacy and minimal side effects. Therefore, the prior art is deficient in the lack of an effective technique using carotid sinus nerve stimulation for epilepsy control. The present invention fulfills this long-standing need and desire in the art.

### SUMMARY OF THE INVENTION

The present invention is directed to carotid sinus nerve stimulation, both in terms of efficacy and apparent associated side effects. Specifically, the carotid sinus nerve) was studied, since it offered the most favorable surgical access and was the most amenable to stimulator implantation.

In one embodiment of the present invention, there is provided a method of treating, controlling or preventing epilepsy and/or influencing neurological function, comprising the steps of attaching at least one electrode to afferents of at least one of a patient's nerve to the carotid sinus, and applying modulating electric signals to at least one of the patient's nerve to the carotid sinus through the at least one electrode.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the cessation of epileptiform activity following stimulation of the carotid sinus nerve: Multi-electrode array recording of ongoing epileptiform activity, stimulation initiation and cessation (on/off), and resolution of epileptiform activity. A continuous concurrent cardiac rhythm strip appears a t the top of the recording. Each long hashmark denotes one second (1 s) on the time (horizontal axis); distance between each of displayed channels (vertical axis) is approximately 3 milliVolts.

### DETAILED DESCRIPTION OF THE INVENTION

The present invention provides a method of treating, controlling or preventing epilepsy, comprising the steps of attaching at least one electrode to at least one of a patient's carotid sinus nerves, and applying modulating electric signals to at least one of the patient's nerve to the carotid sinus through the at least one electrode.

The following examples are given for the purpose of illustrating various embodiments of the invention and are not meant to limit the present invention in any fashion.

### EXAMPLE 1

Materials & Methods Prior to the initiation of this study, approval was received from the Institutional Animal care and Use Committee (IACUC) at the University of Alabama, Birmingham (UAB). Carotid sinus nerve stimulation was studied in five large-sized dogs, with facilities and care provided by the Cardiac Rhythm Management Laboratory a t UAB.

### EXAMPLE 2

Anesthetic Technique

The animals were anesthetized using techniques similar to human seizure patients undergoing temporal lobectomy at this institution. Animals were induced using intravenous thiopental (25 mg/kg body weight). Following intubation, animals were maintained using isoflurane in 100% oxygen (Ohio vaporizer, Division of Airco, Madison, Wis.). End-tidal isoflurane concentrations were maintained at approximately 0.3%. A continuous infusion of intravenous fentanyl (0.2-2.0 micrograms/kilogram body weight/minute) was administered throughout the experiment. Succinylcholine was administered to eliminate any movement during seizure activity or nerve stimulation. Anesthesia was assured by monitoring for changes in systemic arterial pressure or increase in blood pressure during a strong paw pinch. Lactated Ringer's solution was infused at a rate of 5-10 milliliters/kg body weight/hour via the femoral vein throughout this experiment. Animals were mechanically ventilated (Ohio Anesthesia Ventilator, Madison, Wis.), and arterial PCO2 was maintained well within the normal physiological range. A catheter was placed in the femoral artery for continuous measurement of blood pressure and for analyses of arterial blood gases and electrolytes every 30 to 60 minutes. Throughout the experiment vital sign parameters, electrocardiography (ECG), end-tidal CO2, arterial blood gases, and anesthetic drug doses were carefully monitored as noted, and used to direct appropriate care.

### EXAMPLE 3

Surgical Technique

The animal was placed in the supine position, with its head turned 30 degrees contralaterally. The carotid sinus nerve (CSN) was exposed by using a technique analogous to carotid endarterectomy in humans (11): via an initial skin incision along the medial border of the sternocleidomastoid, with deep dissection along this border carried through the platysma to the carotid sheath. The carotid sinus nerve was easily localized just superior to the carotid bifurcation, loosely adherent to the internal carotid artery (ICA) via areolar tissue. This neck dissection was performed bilaterally via separate incisions, and the carotid sinus nerve was marked b y loosely placing a vessel loop around the nerve on each side.

Following bilateral carotid sinus nerve exposure, a left frontoparietal craniotomy was performed using a twist-drill and bone rongeurs; the dura was carefully exposed and then incised in a cruciate fashion to expose the underlying cortex. Extreme care was taken during exposure, as mechanical insult to the brain or the carotid sinus nerve could obscure experimental results.

### EXAMPLE 4

Seizure Induction and Recording

Topical penicillin was then applied to the cerebral cortex: approximately 25 KU of penicillin VK was applied to a 2.5×2.5 square cm area of posterior frontal cortex. A custom-designed multielectrode (2.5 cm×2.5 cm., 504-electrode 21×24 array with 1 mm spacing) electroencephalogram (EEG) array (Wolf PD, Rollins DL, Simpson E V, Smith W M, and Ideker R E., A 528 channel system for the acquisition and display of defibrillation and electrocardiographic potentials. In: Murray A, Arzbaecher R, eds. Proc. Computers in Cardiology. Piscataway, N.J.: The Institute of Electrical and Electronics Engineers, Inc., 1993:125-128) was placed in direct contact to the pial surface of the brain. Recording was performed at a gain of 500, with filters (high-pass 0.05 Hz, low-pass 4 kHz); simultaneous multi-channel recording was ongoing throughout the experiment.

### EXAMPLE 5

Stimulation

Following seizure induction (observed as continuous, non-spontaneously resolving spike activity on EEG), a stimulating electrode was placed around the respective carotid sinus nerve. Placement of the stimulator was performed at varying times following seizure induction. The EEG was closely observed to determine whether seizure resolution occurred, either spontaneously or as a direct result of mechanical stimulation, from stimulator placement onto the carotid sinus nerve. A copper electrode-based customized set-up (Rollins D L, Wolf P, Ideker R E, Smith W M: A MacIntosh-based programmable cardiac stimulator. Abstract in Journal of the American College of Cardiology 15:261A, 1990) was utilized with a constant current stimulator generating square-wave stimulation. Stimulation was conducted at the following parameters: current 10 mA, frequency 40 Hz, pulsewidth 1 ms, time 10 seconds. These settings were extrapolated from previous work related to VNS in the clinical setting (Terry R S, Tarver W B, Zabara J: The implantable neurocybernetic prosthesis system. PACE 14:86-93, 1991.) as well as preliminary work in animals. Stimulation intervals were clearly documented on the EEG display screen, and vital signs, ECG, and overall animal status were all carefully observed contemporaneously. In addition, other vicinal nerves of the cervical region (including the hypoglossal nerve and nerves of the cervical plexus) were stimulated to determine possible effects upon seizure control.

### EXAMPLE 6

Results

All ten carotid sinus nerve specimens were readily found in the five animals evaluated. Exposure of the neck bilaterally and craniectomy were performed in an atraumatic, uncomplicated fashion.

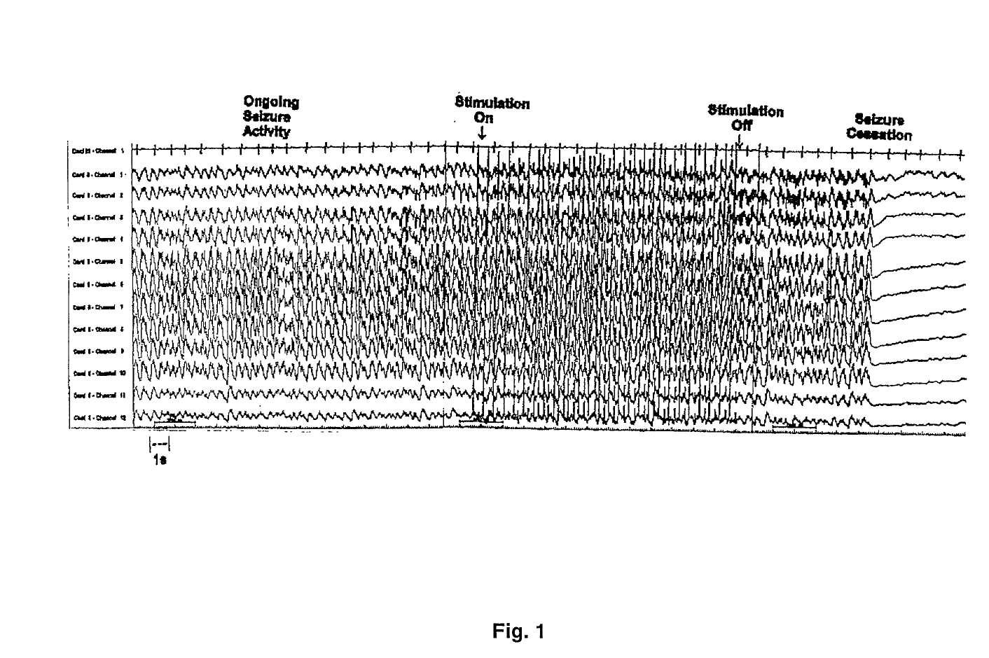

Following topical application of penicillin, spike activity was generally observed within 5-10 minutes; subsequent seizure activity (with continuous, non-resolving spike activity as shown in FIG. 1) was generally observed within 30-45 minutes. Stimulation was performed at varying times (mean 139 seconds, range 1-432 seconds) following onset of seizure activity, to determine both the incidence of spontaneous seizure resolution (i.e. unrelated to carotid sinus nerve stimulation) and the effect (if any) of delayed stimulation. No spontaneous resolution of seizure activity was observed, and no significant change in seizure control was observed with varying delays prior to stimulation. Following carotid sinus nerve stimulation seizure control was apparent (defined as the clear cessation of seizure activity on EEG) in 12 out of 16, or 75% of trials performed. Seizure cessation was noted at a mean of 17 seconds following end of stimulation, with a mean interictal time of 399 seconds. Interestingly, two animals became seizure-free for the duration of our experiment (i.e. for over approximately two hours, prior to which recurrent seizure activity was generally noted at a frequency of approximately 1 seizure per 5 to 10 minutes). An example of seizure activity and resolution following stimulation is shown in FIG. 1.

No apparent seizure control or other EEG change w as noted by the mechanical manipulation of the carotid sinus nerve during placement of the stimulator onto the carotid sinus nerve. Also, no apparent seizure control was noted following stimulation of vicinal nerves (e.g. hypoglossal nerve, nerves of the cervical plexus). Finally, no dangerous side effects were noted (cardiac or otherwise, according to the parameters noted) following either-sided carotid sinus nerve stimulation, and vital signs remained relatively stable throughout the stimulation and post-stimulation periods.

The ninth and tenth cranial nerves share nuclei and neural pathways; hence seizure control, as observed with VNS, should intuitively follow with a branch of ninth cranial nerve stimulation. Although sharing some pathways, distributions of the ninth and tenth cranial nerves are separate in many ways; hence intuitively ninth cranial nerve branch stimulation may not b e associated with the same side effect profile as VNS (especially given the significantly less extensive course of the carotid sinus nerve versus the vagus nerve).

The carotid sinus nerve is surgically the most easily accessible branch of the ninth cranial nerve. Exposure of this nerve entails most of the same initial steps involved in carotid endarterectomy, a procedure routinely performed by many neurosurgeons. Following dissection, the carotid bifurcation is exposed and the carotid sinus nerve identified. A comprehensive study of surgical feasibility of the carotid sinus nerve stimulation in humans, along with nerve dimensions and surrounding landmarks in cadaveric specimens, has shown feasibility of carotid sinus nerve exposure and stimulation capability.

### EXAMPLE 7

Animal Model

The topical penicillin model has been well recognized as a method of inducing focal seizure activity. This model may b e particularly suited to this study, since a large proportion of medically (and most surgically) refractory epilepsy patients appear to suffer from epilepsy which is not primarily generalized. For the purpose of this study, the pentylenetetrazol/strychnine model used by Zabara in initial studies could not be used as this was determined to provide inadequate anesthetic care according to IACUC guidelines.

Anesthesia was administered using isoflurane and fentanyl; this was analogous to human epilepsy surgery performed at this institution. Inhalational agents with epileptogenic potential were avoided to prevent confounding the penicillin model, and mimic anesthesia as for clinical neurosurgical patients.

### EXAMPLE 8

Extrapolation of this Study to Human Patients

The goal of this study was to assimilate and extend the work performed in the field of vagal nerve stimulation. Tenth cranial nerve stimulation has already been studied in great detail with respect to its safety, physiology, and stimulation parameters. Vagal nerve stimulation has evolved significantly following the initial work of Zabara with respect to determination of ideal stimulation parameters in humans, further elucidation of physiologic mechanisms, and characterization of the overall efficacy and side-effect profile in humans. The same detailed analysis and expansion of work in carotid sinus nerve (as well as ninth cranial nerve) stimulation is lacking, and may be promoted by this study.

### EXAMPLE 9

Efficacy of Stimulation

The results of this study indicated a 75% success rate, with 4 instances of stimulation whereupon seizure cessation was not noted. While this overall rate compares favorably with the lower success rate in clinical vagal nerve stimulation, the following reasons may be responsible for the failures. First, the nerve may have been overstimulated, resulting in either a prolonged refractory period or possible damage. The stimulation parameters for this study were extrapolated from vagal nerve stimulation as noted; given the smaller diameter of the carotid sinus nerve in comparison to the vagus nerve, it is possible that slight adjustments in stimulation parameters in the future may help avoid this potential problem. Nonetheless, it is important to note that seizures were controlled in 12 of 16 trials performed; since lack of seizure control was noted to occur at the final trials for a given animal, this supports the compounding effect of repetitive stimulation upon the carotid sinus nerve leading to inefficacy. It is plausible that the seizures themselves became refractory to stimulation, as may happen with treatment failures with vagal nerve stimulation. The occurrence of treatment failures occurring at the final stimulation trials may suggest overstimulation of neural pathways more rostral to the nerve. Since the experimental design called for variation of the time between stimulation (to control for spontaneous seizure resolution), overstimulation of neural pathways due to lesser time intervals between stimulation may have resulted in treatment inefficacy.

Factors such as ideal stimulation parameters, determination of neural pathways responsible for seizure control, and physiological/anatomical analysis of carotid sinus nerve stimulation can be fully characterized by a person having ordinary skill in this art. Further elucidation of these areas could significantly improve upon the few treatment failures.

### EXAMPLE 10

A part of the present study analyzed the anatomy and surgical feasibility of carotid sinus nerve stimulation for seizure control in humans. The carotid sinus nerve appeared the best candidate with respect to both ease of surgical exposure and stimulation capability.

Materials & Methods: Human Cadavers

Fifteen formalin-fixed human cadavers were studied. Eight specimens were male and seven female; ages ranged from 3 weeks to 80 years old (mean 49 years, median 67 years). No pathology was grossly visible in the area of dissection. Specific variables studied in this group appear in Table 1

### EXAMPLE 11

Surgical Exposure

Surgical exposure of the carotid sinus nerve was achieved in a manner analogous to the exposure for carotid endarterectomy. The skin was incised along the palpable medial border of the sternocleidomastoid muscle; dissection was then carried deep through the platysma, medial to the sternocleidomastoid. The carotid sheath was exposed, and the bifurcation of the common carotid artery was clearly identified. The carotid sinus nerve was identified along the internal carotid artery. Specific related parameters were noted, including size of the nerve, position relative to the internal carotid artery, “working distance” (length of the carotid sinus nerve below the mandibular angle available for stimulator placement), and distance relative to nearby localized structures (i.e. the superior edge of the thyroid cartilage, common facial vein, and inferior margin of the superior cervical ganglion). The list of variables analyzed in this study appears in Table 1.

TABLE 1

List of variables
Nerve diameter
Position relative to internal carotid artery
Working distance (length of carotid sinus nerve inferior to
mandibular
angle available for stimulation)
Distance from nearby localizable landmarks: superior edge of
thyroid cartilage,
common facial vein, inferior margin of the superior cervical
ganglion

### EXAMPLE 12

Results

All cadavers exhibited the carotid sinus nerve bilaterally. The mean diameter was 1.0 mm., with no significant difference between left versus right sides (p<0.05). The carotid sinus nerve was always of equal caliber throughout its course, from origination at the extracranial main ninth cranial nerve trunk to its termination on the carotid sinus. The nerve terminated at the carotid bifurcation in all cases. The carotid sinus nerve occurred as a single branch, loosely adherent to the internal carotid artery in all specimens examined. With respect to the internal carotid artery, the carotid sinus nerve coursed anteromedially in 9 of 15 cases (60%), anteriorly in 5 of 15 cases (33%), and anterolaterally in 1 of 15 cases (7%). The nerve always appeared below the mandibular angle, with a mean “working distance” for stimulator implantation of approximately 1.5 cm. (range 6 to 30 mm.) and no significant difference between sides (t-test: P<0.05).

The visible termination of the nerve at the carotid sinus was also evaluated with respect to three surrounding structures: the superior edge of the thyroid cartilage (STC), common facial vein (CFV), and inferior margin of the superior cervical ganglion (SCG). The nerve terminated at the level of superior edge of the thyroid cartilage in 64% of the specimens, below superior edge of the thyroid cartilage in 30%, and superior to superior edge of the thyroid cartilage in only 6% (with no difference between sides or gender, t-test: P<0.05 for each); the superior edge of the thyroid cartilage was 5.4 mm (mean) below the nerve termination (ranging from 35 mm below to 33 mm above it). The common facial vein was found in 20 of 30 sides; with respect to common facial vein, the nerve terminated at the level of common facial vein in 65%, inferior to common facial vein in 22%, and superior to common facial vein in only 13% (with no difference between sides or gender, t-test: P<0.05 for each); the common facial vein was 3.0 mm (mean) above the nerve termination (ranging from 10 mm below to 15 mm above it). With respect to inferior margin of the superior cervical ganglion, the CSN termination was consistently inferior, ranging from 6 to 30 mm. (mean 14.7 mm.).

A summary of findings appears in Table 2.

TABLE 2
Summary of findings

| Variable | Value |
|---|---|
| Nerve diameter (mm) | mean 1.0, range [0.5 to 1.5] |
| Position relative to | anteromedial (9/15 or 60%) |
| internal carotid artery: | anterior (1/15 or 7%) |
| | anterolateral (5/15 or 33%) |
| Working distance (mm) | mean 15, range [6 to 30] |
| Distance (mm) | |
| above carotid bifurcation: | |
| CFV | mean 3.0, range [−10 to +15] |
| STC | mean −5.4, range [−35 to +33] |
| SCG | mean 16.6, range [6 to 30] |

Key to abbreviations:
CFV = common facial vein,
STC = superior border of thyroid cartilage,
SCG = inferior margin of superior cervical ganglion.

### EXAMPLE 13

Functional Anatomical Basis

The carotid sinus and tenth cranial nerves share some nuclei and potentially other neural pathways. The tenth cranial nerve has been more extensively studied with respect to vagal stimulation for seizure control. However, since the course of the carotid sinus nerve is not as extensive as the vagus, particularly with respect to cardiac innervation, carotid sinus nerve stimulation should be associated with fewer (if any) cardiac side effects (such as the life-threatening dysrhythmias associated with right-sided vagal nerve stimulation).

### EXAMPLE 14

Clinical Anatomical Basis

Clinical studies of the carotid sinus nerve in humans have been largely anectodal, usually describing effects following manipulation of the nerve. Transient hypertension and tachycardia or syncope have been reported. Anatomical studies have largely involved animal models, although occasional study of ninth cranial nerve anatomy in human cadavers as well as intraoperative dissection has been described in the literature. The study by Goodwin et al. examined ninth cranial nerve anatomy in four cadaveric specimens and five patients intraoperatively during regional otolaryngological procedures; however, this study focused largely on the ninth nerve trunk and did not address the carotid sinus nerve nerve specifically in great detail.

The goal of this anatomic study was the determination of caotid sinus nerve stimulation feasibility in humans. The ability of successful implantation is particularly encouraging in light of earlier work regarding successful carotid sinus nerve stimulation for medically refractory hypertension control. This study reported successful long-term implantation of a stimulator around the carotid sinus nerve in several patients.

### EXAMPLE 15

Ease of Dissection and Implantation

The procedure was performed in the same manner as for carotid endarterectomy, which is routinely performed by many neurosurgeons worldwide. Dissection and localization may be aided by surrounding localizing landmarks. In this study, the common facial vein and superior edge of the thyroid cartilage were evaluated and found to be helpful in localizing the carotid bifurcation, as h as been previously described. The findings of this study would suggest that the common facial vein more closely approximated the bifurcation, although it was absent in several cases; the superior edge of the thyroid cartilage was more consistently present, although more variable in its relationship to the bifurcation. Finally, the relationship of the inferior margin of the superior cervical ganglion to the bifurcation was analyzed to determine the likelihood of sympathetic fiber association at the CSN takeoff. It was postulated that sympathetic contributions towards the internal carotid artery would unlikely be affected by carotid sinus nerve stimulation, as the inferior margin of the superior cervical ganglion generally appeared superior to the bifurcation.

Implantation feasibility was mainly addressed b y determination of (1) nerve diameter, and (2) working distance inferior to the mandibular angle. Once isolated and dissected free of loose connective tissue approximating this nerve to the internal carotid artery, measurements of the nerve were taken. With a mean diameter of 1.0 mm. and minimum of 0.5 mm., the carotid sinus nerve stimulation was deemed feasible under direct observation Additionally, with the bifurcation consistently inferior to the mandibular angle, ample working distance was available for stimulator placement. Further support for stimulator implantation feasibility is found by the previously described carotid sinus nerve stimulation series by Schwartz et al., wherein eleven patients suffering from medically refractory hypertension were treated with this technique.

The findings of this study indicate that surgical exposure of the carotid sinus nerve is feasible, and that the nerve is amenable to stimulator placement. Dimensions of the nerve and its location relative to the mandible further support stimulation ability. Finally, relationships to surrounding structures, some of which may aid in localization, have been presented. It is hoped that the success of this study, in conjunction with the efficacy of carotid sinus nerve stimulation in seizure control (as demonstrated by the animal model experiments disclosed herein), offer a method for epilepsy control in humans.

The results of this study have shown that: Carotid sinus nerve stimulation, performed via stimulation of the carotid sinus nerve, offered seizure control in the majority of trials. No significant side effects were observed for either left- or right-sided stimulation (which differs from vagal nerve stimulation, which is contraindicated for the right side). A potentially novel method of therapy may exist for medically (and presently surgically) refractory epilepsy patients.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of treating, controlling or preventing epilepsy and/or influencing neurological function, comprising the steps of attaching at least one electrode to afferents of at least one of a patient's carotid sinus nerves, and applying modulating electric signals to at least one of the patient's carotid sinus nerves through the at least one electrode, so as to treat, control, or prevent epilepsy.

2. The method of claim 1, including but not limited to the following modulating electric signals: frequency of about 5 to about 300 cycles per second, at a constant current of between about 0.5 and about 20 milliamperes, and a pulse width of between about 0.1 and 1 millisecond.

3. The method of claim 1, wherein patients experiencing symptoms of an epileptic attack are treated by a process comprising the steps of:
detecting a physiological symptom associated with onset of a epileptic attack in a patient under treatment, and
selectively applying said modulating electric signals in response to detection of such symptom, as a programmed electrical stimulus to at least one of the patient's carotid sinus nerves, for modulating electrical activity of preselected fibers of said nerve, in a manner to alleviate the epileptic attack.

4. The method of claim 1, wherein said electric signals are applied repeatedly over a period of time to thereby prevent or control such seizures.

5. The method of claim 1, wherein stimulation was conducted at the following parameters: current 10 mA, frequency 40 Hz, pulsewidth 1 ms, time 10 seconds.

6. The method of claim 1, wherein the signal is applied utilizing a plurality of electrodes.

7. The method of claim 1 including the step of repeatedly applying said signal over a period of time.

8. The method of claim 1, whereby either external or internal instruments are used for carotid sinus nerve stimulation or recording.
